(19) United States
(12) Patent Application Publication
(10) Pub. No.: US 2018/0028390 A1
(43) Pub. Date: Feb. 1, 2018

(54) ORTHOPEDIC SYSTEM AND METHOD FOR ITS CONTROL

(71) Applicant: OTTO BOCK HEALTHCARE PRODUCTS GMBH, Wien (AT)

(72) Inventors: Hans DIETL, Gablitz (AT); Roland AUBERGER, Wien (AT); Michael Friedrich RUSSOLD, Wien (AT); Christian HOFER, Wien (AT)

(73) Assignee: OTTO BOCK HEALTHCARE PRODUCTS GMBH, Wien (AT)

(21) Appl. No.: 15/662,039

(22) Filed: Jul. 27, 2017

(30) Foreign Application Priority Data
Jul. 29, 2016 (DE) 102016114075.9

(51) Int. Cl. A61H 3/00; A61F 2/68; A61N 1/05; A61N 1/04; A61N 1/36

(52) U.S. Cl. CPC A61H 3/00; A61N 1/0452; A61N 1/36003; A61N 1/0504; A61F 2/68; A61F 2002/5039

(57) ABSTRACT

An orthopedic system having at least one stimulation electrode for activating at least one muscle, an orthotic or prosthetic device, which may be placed on a patient's body and secured there with at least one joint by which a proximal and a distal component of the orthotic or prosthetic device are connected to each other in swivelling fashion, at least one adjustable resistance device which is arranged between the distal component and the proximal component and with which a motion resistance against a swivelling of the proximal component relative to the distal component is adjustable, sensors for detecting of forces positions, accelerations and/or torques, and a control device which is coupled to the sensors and the resistance device, processes sensor values of the sensors and adjusts the resistance device in dependence on the sensor values.

### TECHNICAL FIELD

The present disclosure relates to an orthopedic system having at least one stimulation electrode for activating at least one muscle, an orthotic or prosthetic device which may be placed on a patient's body and secured there, and at least one joint by which a proximal and distal component of the orthotic or prosthetic device are connected to each other in swivelling fashion. The present disclosure also relates to a method for control of such an orthopedic system in which at least one muscle of the patient is activated via the at least one stimulation electrode.

### BACKGROUND

U.S. Pat. No. 7,403,821 discloses a method for producing a dorsal flexion of a patient's foot, in which stimulation electrodes and signal detection electrodes are arranged on at least one peripheral nerve of the thigh. Neural signals are received and processed by the electrodes in order to determine a movement or an action indicating the time of a heel push or a heel lift during walking. Depending on the action detected, the stimulation electrode will be activated to produce a dorsal flexion of the patient's foot.

EP 1 257 318 B1 discloses a device for producing a dorsal flexion and for stimulating a motor nerve fibre with features for receiving and processing the nerve signals detected and for generating stimulation signals. The device comprises a combined detection and stimulation electrode device and is designed as a combined electrode. The device operates to switch each combined electrode between a detection state and a stimulation state. The combined detection and stimulation electrode device is implantable in design.

Furthermore, it is known from the prior art how to equip a prosthetic device, such as one for a lower limb, with a resistance device and perform an adapting of the movement resistances in dependence of sensor values ascertained. Such a device and such a method are known for example from DE 10 2009 052 887 B1.

People who require prosthetic devices may have additional impairments in the voluntary activation of the muscles. The same holds for those who depend on an orthotic for the lower limbs, for example due to accidents, neuronal damage, or other impairments of the voluntary activation. Furthermore, the lack of sensory feedback may impair the mobility of the patients so that a deteriorated walking ability or even a fundamental impairment of the general function of the lower limb may develop. The cause of this impairment may be paralysis, muscle defects after medical procedures or oncological diseases, stroke, other cerebral disorders or the like.

Furthermore, prosthetic or orthotic devices exist with external, frequently electric motorized drive units in order to make up for the motoric deficit of the muscles. Such active prostheses or orthotics are very heavy, require a high energy consumption, and generally produce annoying noises. One example of an active prosthesis is described in U.S. Pat. No. 5,246,465.

### SUMMARY

One problem which the present disclosure proposes to solve is to provide an unobtrusive orthopedic system and a method for its control with which the motion ability of a patient, especially the walking ability of a patient, may be improved. Advantageous embodiments and modifications of the present disclosure are disclosed in the description and figures.

One embodiment of an orthopedic system in accordance with the present disclosure includes at least one stimulation electrode for activating at least one muscle, an orthotic or prosthetic device which may be placed on a patient's body and secured there, and at least one joint by which a proximal and a distal component of the orthotic or prosthetic device are connected to each other in swivelling fashion. The orthopedic system also includes at least one adjustable resistance device which is arranged between the distal component and the proximal component and with which a motion resistance against a swivelling of the proximal component relative to the distal component is adjustable, a plurality of sensors for detecting of forces, positions, accelerations and/or torques, and a control device which is coupled to the sensors and the resistance device, wherein the control device processes sensor values of the sensors and adjusts the resistance device in dependence on the sensor values. Unlike with motor operated orthotics or prostheses, in the orthopedic system according to the present disclosure includes a stimulation electrode to stimulate a muscle, for example by an electric pulse, so that the muscle contraction that follows has an intensified dynamic of movement or initiates an overall movement of the limbs or the residual limbs. The activated muscle results in an increase in movement energy within the orthopedic system, which leads to a corresponding movement of the distal and/or proximal component of the orthopedic system.

The adjustable resistance device provides control and/or regulation of the movement in dependence on sensor values, which are transmitted to a control device, which in turn is coupled to the resistance device, so that the desired movement pattern may be accomplished. The orthopedic system is operable without a costly motor drive unit and without a comparably heavy energy accumulator to put energy into the system by electrically activating the muscles. The movement of the orthotic or prosthetic device is controlled via the adjustable resistance of the resistance device in order to accomplish an approximation or adaptation to the desired or intended movement pattern.

The stimulation electrode may be designed as a surface electrode or as an implant. The choice of the electrode form depends on the muscle being activated, the course of a nerve being stimulated, or the type and manner of muscle activation. If the muscle is activated by the excitation of a nerve which is still present, running relatively close to the skin surface, the design of the stimulation electrode as a surface electrode may be advantageous, since such an electrode is easy to put in place and does not require any implantation cost. Surface stimulation electrodes may be affixed relatively easily to the skin surface, but they have the potential drawback that the locating and thus also the activating of the particular nerve may be imprecise. The design of the electrode as an implant has the advantage of a permanent and precise matching of the electrode with the particular nerve or muscle when this is being directly activated. The implanted electrode may be designed as a so-called cuff electrode and lie like a sleeve around the nerve being excited and leading to the muscle being stimulated, when the muscle or the nerve supplying it is activated.

The implanted stimulation electrode may be designed as a percutaneous stimulation electrode, especially for a trial usage in which the electrode is implanted in direct proximity to the nerve branches being excited. Besides the design as a cuff electrode, the electrode may also be designed as electrode plates with several contacts or in some other way. The arrangement is done in immediate proximity to the nerve branches innervating those muscles which are responsible for the particular movement, such as those for a dorsal flexion or extension of a foot, a shin flexion or extension, or a hip flexion or extension. It is likewise possible to arrange an orthotic or prosthetic device on an upper limb and arrange a corresponding stimulation electrode in immediate proximity to those nerve branches which innervate the muscles being activated. An electrode line extends from the point of stimulation on the nerve branches through the skin to an external pulse generator, e.g., a so-called stimulator. In addition, implanted stimulation electrodes may also be designed as a hybrid implant, in which a cable leads from the electrode to a likewise implanted pulse generator or a receiver which is placed beneath the skin surface. In this case, there is no penetration of the skin. An energy supply for the electrode and a control of the electrode pulses occurs through a sending and receiving unit placed directly on the skin surface above the pulse generator, which is designed to transmit energy in the direction of the pulse generator and information to the pulse generator and from the pulse generator to the sending and receiving unit.

Another variant of the design of the electrode as an implant includes implanting the electrode, a pulse generator, the energy supply and the control device together. Such an implantable module may work independently and needs no other components outside the module, once the control unit is set up. The energy supply for the control device and the generating of a pulse occurs either through a battery or a storage cell. A connection of the implant to the outside may be wireless in order to make adjustments or read out data.

One variant of the present disclosure includes the stimulation electrode coupled to the control device, either by wire, wirelessly, or by integration of the control device in the stimulation electrode. Through the control device, the point in time, the type of pulse, the pulse duration and the pulse strength are established for activating or stimulating the nerve or the muscle directly.

The orthotic or prosthetic device may be associated with an energy accumulator, by which kinetic energy of the orthotic or prosthetic device is storable and returnable to the orthotic or prosthetic device in a controlled manner. It is possible to transform kinetic energy from the orthopedic system using the energy accumulator, for example, when excess movement energy needs to be dissipated, while the storage of the kinetic energy does not dissipate the kinetic energy, in particular, does not convert the kinetic energy completely into heat. Instead, it is possible to return the kinetic energy once more to the orthopedic system after an appropriate transformation, once the kinetic energy has been stored in another form of energy, such as potential energy by the deformation of a spring, by charging of a pneumatic or hydraulic pressure accumulator, or by transformation into electrical energy. It is thus possible to assist the orthotic or prosthetic device in its movement, for example, to assist a flexion or extension of the components relative to each other, for example, to assist in standing up or lifting an object. Alternatively, a transformation occurs especially when an increased resistance to a movement needs to be applied, for example, when decelerating, when sitting down or when walking in stance phase flexion.

The energy accumulator may be designed as a spring accumulator, pressure accumulator, or accumulator of electric energy, wherein the orthotic or prosthetic device may be associated with a drive unit in order to transform the stored energy into kinetic energy once more and enable a flexion or extension of the distal component relative to the proximal component.

The orthotic or prosthetic device representing an orthopedic device or an orthopedic system may be designed as a lower limb orthosis or prosthesis. The resistance device may be formed as, for example, a mechanical brake, a fluid damper, a magnetorheological damper, or a generator, while the resistance device may be adjusted by an adjusting device, such as an actuator, which is coupled to the control device, in dependence on the sensor values in order to provide an adapted resistance. The resistance may be increased or decreased, and the adjustment may be done in dependence on the detected sensor quantities, such as forces, positions of the distal and/or proximal components in space or relative to each other, accelerations which the distal and/or proximal components perform or perform relative to each other, and/or torques acting on the distal and/or proximal components.

A method according to the present disclosure for the control of an orthopedic system, such as is described above, includes activating at least one muscle of the patient via the at least one stimulation electrode. Through sensors which are arranged on the orthotic or prosthetic device or on the patient, it is possible to detect forces, positions, accelerations and/or torques. The forces, accelerations and/or torques may be acting on the components of the orthotic or prosthetic device and/or on the limbs or stumps on which the orthotic or prosthetic system is secured. The positions of the components of the orthotic or prosthetic device as well as the respective limbs may be determined in relation to a fixed reference quantity, such as the direction of gravity, or in relation to each other. The sensor values detected by the sensors are relayed to the control device, which operates to change the resistance in the resistance device in dependence on the sensor values. The control device may relay a corresponding signal to an adjustment device, which is coupled to the resistance device, in order to decrease or increase the resistance of the resistance device. For example, valves of the resistance device and/or the adjustment device may be opened or closed in order to produce a fluid flow with a decreased or increased flow resistance. Likewise, magnetic excitation may be used to change a magnetorheological damper in the resistance produced by the resistance device. If a mechanical brake is used as the resistance device, an increasing of the braking force, for example, a pressing by brake linings, or a decreasing of a pressing force of a wrap spring brake may result in an adapting or adjusting of the resistance provided by the resistance device. When the resistance device is designed as a generator, an increased resistance to a rotation may be provided by excitation of coils.

One modification of the present disclosure includes activating muscles which act antagonistically to each other using at least two stimulation electrodes delayed in time, in order to produce a flexion and extension of the limb connected to the muscles. The time delay may be permanently programmed in the control device. Alternatively, the mutual stimulation is controlled via sensors, which are coupled to the control device and produce a corresponding stimulation in dependence on the movement parameters or loading parameters detected.

One modification of the present disclosure includes a drive unit which is associated with the orthotic or prosthetic device assisting a swivelling movement of the proximal component relative to the distal component, for example, to ease the lifting of an object or to help a patient in standing up.

The movement energy from the activated muscles during a decelerating of the movement of the orthotic or prosthetic device may be stored in an energy accumulator and returned delayed in time for the driving of the orthotic or prosthetic device. In this way, it is possible that the muscles do not need to be activated during each movement of the orthotic or prosthetic device or the activation may occur at a lower excitation level. Furthermore, it is possible by transforming the kinetic energy into electric energy to use the kinetic energy for the power supply of the stimulation electrodes and the control device.

A muscle activation may occur in dependence on a force exerted on a limb or a component of the orthotic or prosthetic device or a torque exerted on a limb or a component of the orthotic or prosthetic device. Depending on the size of the loading, it is thus possible to accomplish an adapted muscle activation. The force or torque exerted on the orthotic or prosthetic device or the forces or torques exerted on the limbs represent movement indicators providing feedback as to whether a muscle activation should occur and by how much.

A corresponding muscle activation may occur in dependence on an acceleration performed by a limb or a component of the orthotic or prosthetic device and/or in dependence on the position of a limb or a component of the orthotic or prosthetic device in space or relative to each other. It is likewise possible to base the muscle activation on a combination thereof, for example, based on a torque exerted on a component based on the current position of the component in space or relative to another component.

An activation of the hip muscles and/or the thigh muscles may occur in order to assure an improved walking ability and mobility of a patient.

### BRIEF DESCRIPTION OF DRAWINGS

Illustrative embodiments of the invention are explained in more detail below with reference to the attached figures, in which:

FIG. 1 illustrates an embodiment of the present disclosure as an orthotic device.

FIG. 2 illustrates an embodiment of the present disclosure as a prosthetic device.

FIG. 3 illustrates schematically a control device for use with the orthotic and prosthetic devices of FIGS. 1 and 2.

FIG. 4 is a flow diagram illustrate steps of a method in accordance with the present disclosure.

### DETAILED DESCRIPTION

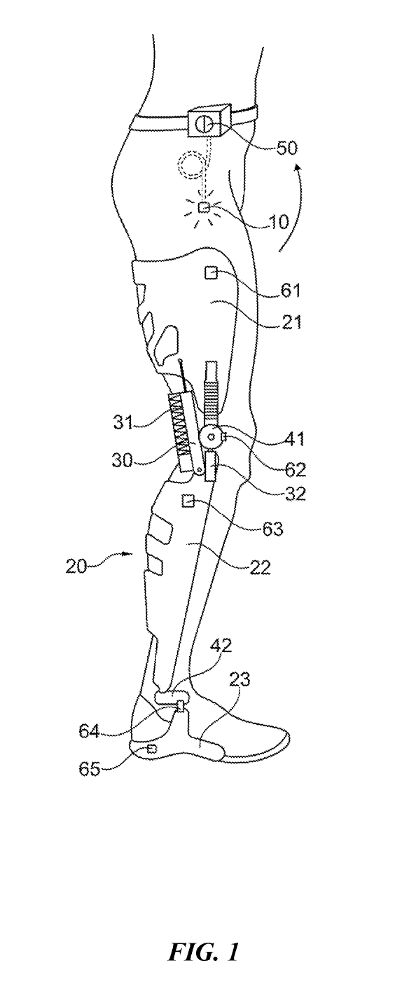

FIG. 1 shows an orthopedic system with a stimulation electrode 10, which is configured as an implantable electrode and implanted in the sample embodiment shown. The stimulation electrode 10 operates to activate a hip flexion muscle or a group of muscles which bring about a hip flexion. The stimulation electrode 10 in the sample embodiment shown is configured as a percutaneous stimulation electrode in the form of a cuff or in the form of electrode plates with several contacts in direct proximity to nerve branches. The stimulation electrode 10 innervates the at least one muscle or the group of muscles which are responsible for the hip flexion. The stimulation electrode 10, besides activating the muscle via the nerve or nerves supplying it may also activate the muscle directly, for which a direct coupling of the electrode to the muscle is necessary and is so provided.

In the percutaneous stimulation electrode 10, an electrode line may lead from the stimulation site through the skin to an external pulse generator or the control device 50, which typically is not implanted but secured to the patient outside their body. The coupling of the electrode line to the control device 50 may occur by a detachable connector or a magnetic contact surface, so that a separation of the stimulation electrode 10 from the control device 50 is possible.

The stimulation electrode 10 may also be designed as a hybrid implant, in which a cable leads from the stimulation electrode 10 to an implanted pulse generator or a receiver surface, which is arranged in the skin surface. The power supply as well as the control of the electrode for activating the muscle occurs either directly or via the nerve supplying the muscle through the external control device 50, which has a sending and receiving unit arranged directly on the skin surface above the pulse generator or the contact surface, which is coupled by cable or radio to the control device 50. The transmittal of energy and information occurs through the sending and receiving unit.

An alternative to the arrangement of the control device 50 externally on the patient is for the electrode together with the pulse generator, the power supply and the control device to be combined as a full implant which works autonomously. A wireless connection from the implant to the outside may be provided in order to be able to make adjustments or read out data. The activating of the muscle may occur either directly or via the stimulation of the nerve supplying the muscle through the electrode. The full implant may be provided at the same time with a sensor device in order to obtain sensor data as to the activation of the muscle and/or the position of the implant in space or forces and/or torques acting on it.

The orthotic device 20 of the orthopedic system may be designed in the form of a KAFO (knee ankle foot orthotic), e.g.,, an orthotic extending from the thigh to the foot and spanning the knee joint and the ankle joint. The orthotic device 20 shown in FIG. 1 has a proximal component 21 in the form of a thigh splint or a thigh shell, which is pivotably coupled by an orthotic knee joint 41 to a distal component 22 in the form of a shin splint or shin shell. Further distally, a foot component 23 in the form of a foot shell or a foot splint is arranged in/on the shin component 22, being connected by an orthotic ankle joint 42 to the shin component 22. The shin component 22 may also constitute the proximal component if no thigh component 21 is secured to it. The orthotic device 20 would then only be an AFO (ankle foot orthotic), wherein a resistance device (not shown) may be arranged between the foot part and the foot component 23 as the distal component and the proximal component 22 being the shin splint or shin shell.

A resistance device 30 may be arranged between the proximal component 21 or thigh splint or thigh shell and the distal component 22 or shin splint or shin shell and secured to a component 21, 22. The resistance device 30 may provide a resistance to flexion or extension of the proximal component 21 relative to the distal component 22. The resistance device 30 may also be designed as a blocking device and prevent any swivelling of the distal component 22 relative to the proximal component 21. An adjusting device (not shown) associated with the resistance device 30 may operate to adjust or increase the resistance in the resistance device 30 to a maximum so that no relative movement about the joint axis of the orthotic knee joint 41 is possible in the context of the loading which usually occurs when using a lower limb orthotic.

The resistance device 30 may be designed as a fluid damper, especially as a hydraulic damper, or a mechanical brake, a magnetorheological resistance device or a generator. The resistance device 30 is typically positioned between the components 21, 22 in order to provide an adapted resistance to a swivelling movement, but may be positioned at other locations to provide resistance to movement between other components of the orthotic device 20.

The resistance device 30 may be associated with an energy accumulator 31, which is shown schematically and may be designed as a spring accumulator or a pressure accumulator. The energy accumulator 31 may be configured as an accumulator of electric energy, in order to transform kinetic energy into electric energy. In the design of the energy accumulator 31 as a mechanical energy accumulator, the kinetic energy expended in a flexion and/or extension of the components 21, 22 relative to each other is transformed into potential energy. The energy accumulator 31 may dispense stored energy in a timed manner via either the control device 50 or via a separate orthotic control unit coupled to the control device 50 in order to assist in a flexion or extension of the components 21, relative to each other. The schematically represented motor drive unit 32 may assist with dispensing the stored energy. The drive unit 32 may be supplied with energy from the energy accumulator 31 and brings about or at least assists a swivelling movement of the components 21, 22 relative to each other in one direction or the other.

A corresponding design with a resistance device 30, energy accumulator 31, and drive unit or motor drive unit 32 may also be arranged between the shin component and the foot component 23 in order to provide a desired or required resistance to a dorsal flexion or dorsal extension of the foot, or else to bring about a corresponding driving power and a swivelling movement via the energy accumulator 31 and the drive unit 32. Instead of a swivelling of the shin component 22 relative to the thigh component 21, there may be a swivelling of the foot component 23 relative to the shin component 22. In one embodiment, multiple (e.g., two) resistance devices 30 are provided with one or two energy accumulators 31 and one or two drive units 32 in order to span both joints 41, 42, drive and brake several components, and enable an improved walking ability or an adapting of the walking ability to a desired walking ability.

In the sample embodiment shown, there are five sensors 61, 62, 63, 64, 65 arranged in the orthotic device 20. The sensors 61, 62, 63, 64, 65 are coupled to the orthotic device 20 and the control device 50. Other embodiments may include different numbers of sensors to provide the desired feedback related to, for example, at least one of forces, positions, accelerations and torques.

A first sensor 61 is arranged on the thigh component 21. The first sensor 61 may be coupled wirelessly to the control device 50 and operable to detect the position or angle of the thigh component 21 and thus also the thigh when the orthotic device 20 is being worn. The first sensor 61 may be designed as a gyroscope or other positional sensor in order to detect the orientation of the thigh component 21 and the thigh relative to the vertical (e.g., a vertical plane or vertical axis, or a vertical direction).

A second sensor 62 is arranged in the area of the orthotic knee joint 41. The second sensor 62 may be designed as an angle sensor in order to determine the angle of the thigh component 21 relative to the shin component 22. The second sensor 62 may be designed as an incremental sensor or an absolute sensor. The second sensor 62 may be designed as an optical sensor or be based on a magnetic principle, such as a Hall effect sensor.

A third sensor 63 is arranged in the area of the orthotic knee joint 41 on the shin component 22, e.g., the component which is secured distally and pivotably to the thigh component 21 via the orthotic knee joint 41. The third sensor 63 may be designed either as a position sensor analogously to the first sensor 61 or as a torque sensor or a combination of the two, in order to detect the absolute position of the shin component 22 and/or the loads acting on the shin component 22. It may be possible to arrange another sensor component (e.g., third sensor 63) in the second angle sensor 62 for determining the knee angle, for example, in order to detect the torques or forces acting in the knee joint 41.

Distally to the shin component 22 there is secured a foot component 23 able to pivot by the ankle joint 42. The ankle joint 42 is located at the height of the natural ankle joint, and accordingly the orthotic knee joint 41 is located in the area of the natural knee joint or the natural knee joint axis. The foot component 23 includes a sole component that extends underneath the foot to guide and assist the foot in the dorsal flexion and plantar flexion direction. A fourth sensor 64 is arranged on the ankle joint 42, which detects the angle of the foot component 23 relative to the shin component 22 and/or the torques acting about the ankle joint 42, so as to obtain feedback as to the current loading and the movement situation or walking situation.

A fifth sensor 65 is positioned on the foot component 23 to detect the heel impact or heel contact. The fifth sensor 65 may be a force sensor 65 or pressure sensor and may be positioned in a heel area of the foot. The fifth sensor 65 may provide feedback as to the walking situation, the course of the walking situation and the current movement situation, and may be referred to as a heel sensor. If only a slight loading is detected by the fifth sensor 65 unchanged over a lengthy period of time, it may be assumed that the wearer of the orthotic device 20 is in a sitting posture. In the event of a changing load value detected by the fifth sensor 65 in the heel region with a complete relieving of the load, a walking may be inferred. The magnitude of the loading, the course of the loading and the interval of loading and load relieving as detected at least in part by the fifth sensor 65 may lead to conclusions as to the type of movement and the walking speed.

All values of the sensors 61-65 are relayed to the control device 50 and/or a separate second control device (not shown), which undertake(s) a changing of the flexion resistance and/or extension resistance in the knee joint 41 and/or the ankle joint 42 on the basis of the sensor data. Likewise, the control device 50 may operate to bring about the storage of energy in the energy accumulator 31 and/or the dispensing of energy from the energy accumulator 31 to the drive unit 32, in order to bring about a movement assistance as well as an energy accumulation in order to decrease the kinetic energy expended due to, for example, an activated hip flexion muscle.

The control device 50 may also contain or be coupled to a stimulator for generating a stimulation pulse or pulses.

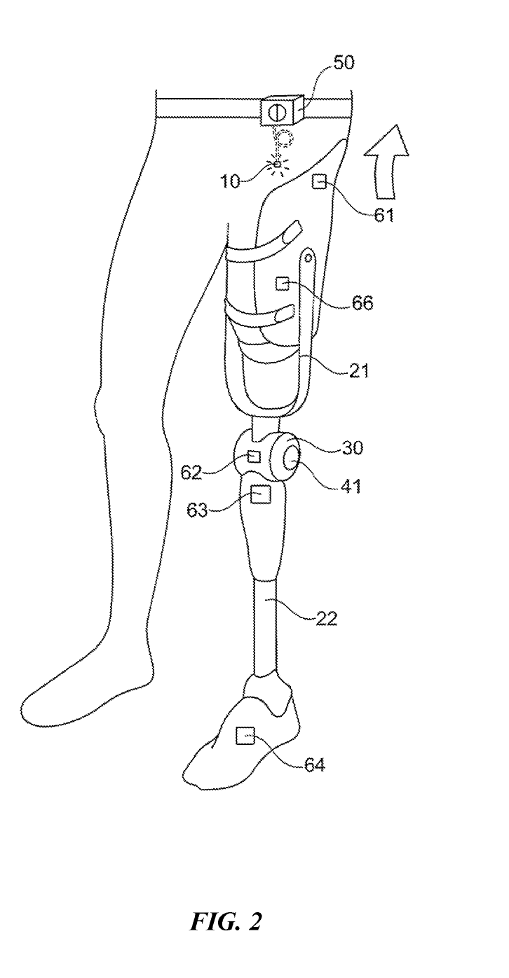

Another embodiment of the present disclosure is represented in FIG. 2, where there is a prosthetic device instead of the orthotic device shown in FIG. 1. FIG. 2 illustrates an external control device 50, which is secured outside the body on a belt worn by the patient. A first sensor 61 (e.g., a position sensor) is coupled to a thigh component in the form of a thigh socket 21, which is connected by a clasp to a prosthetic knee joint 41. The first sensor 61 in the sample embodiment shown is coupled by a cable to the control device 50. A separate position sensor 66 positioned on a thigh socket 21 may be coupled by radio to the control device 50 in order to provide more precise information about the current position of the thigh socket 21 as well as the thigh stump.

A prosthetic knee joint 41, which pivotably connects the proximal thigh component 21 to the distal shin component 22, is arranged at the distal end of the thigh component 21. Inside the prosthetic knee joint 41 is arranged an adjustable resistance device 30, which may be designed as, for example, a mechanical brake, a hydraulic damper, a pneumatic damper, a magnetorheological damper, or a generator. An energy accumulator in the form of, for example, a spring, a pressure vessel, or a storage cell, may likewise be integrated in the prosthetic knee joint 41. Also, a drive unit, such as a spring drive or an electric motor, may be integrated in the prosthetic knee joint 41 in order to both store energy and transform energy during a braking of the shin component 22 relative to the thigh component 21, for example, rather than the energy be converted into heat. The drive unit may store the energy usefully so that the energy may be provided once again via the drive unit for the extension or flexion of the shin component 22 relative to the thigh component 21.

A second sensor 62 is arranged on the prosthetic knee joint 41 for detecting the knee angle and/or the torque acting on the knee joint 41. Distally to the prosthetic knee joint 41 is arranged a third sensor 63 on the shin component 22, which may detect the position of the shin component 22 relative to the thigh component 21 or in absolute terms relative to the vertical. A fourth sensor 64 is positioned on the prosthetic foot for detecting the axial force acting on the prosthetic foot or a torque. All sensors 61, 62, 63, 64, 66 are coupled to the control device 50, which in turn is coupled to the stimulation electrode 10 for stimulating at least one muscle, e.g., the hip flexion muscle in the sample embodiment shown. Furthermore, the control device 50 may also control the resistance device 30 inside the prosthetic knee joint 41 in order to provide an increased or decreased flexion or extension resistance in dependence on the sensor values provided by sensors 61, 62, 63, 64, 66.

Both the orthotic device 20 and the prosthetic device 20 function by the principle of actuating at least one stimulation electrode 10 via the control device 50 and this is provided with a stimulation pulse in order to excite the particular muscle to contract. In the sample embodiment shown, a hip flexion muscle or a hip flexion muscle group is activated by the stimulation electrode either directly or via the nerve supplying the muscle group, so that a hip flexion corresponding to the arrows shown in FIGS. 1 and 2 is activated. Through the hip flexion, an energy pulse is put into the orthotic or prosthetic device 20, by which the shin component 22 or the shin component 22 is moved forward in the walking direction. Accordingly, the foot component 23 or the prosthetic foot is also moved forward, so that an extension movement occurs in the orthotic or prosthetic knee joint 41. Since the control of the muscle pulses may not always be sufficiently precise, the flexion movement and extension movement of the distal component 22 or the shin component relative to the proximal component 21 or thigh component is corrected via the resistance device 30, the resistance device 30 being adjusted in dependence on one or more sensor values from one or more of the sensors 61-66. If too much energy is put into the orthotic or prosthetic device 20 by the muscle activation, an increased resistance may be provided through the resistance device 30. Accordingly, if less energy or too little energy is provided due to a weaker muscle contraction, the resistance is reduced accordingly or an end stop is adjusted so that a harmonious walking pattern or a given walking pattern is achieved.

The orthotic or prosthetic device 20 may cooperate synergistically or antagonistically with the stimulated muscles. Besides the stimulation of the hip flexion muscles, a stimulation of the hip extension muscles may also occur. It is likewise possible to activate the hip flexion through the musculus sartorius, the musculus rectus femoris and the musculus iliacus, as well as the musculus psoas. Then, to counteract the hip flexion movement, the hip extension may be activated through the musculus gluteus maximus. A time-controlled activation of the respective muscles may accomplish a coordinated hip flexion and/or hip extension. Likewise, on the basis of the sensor values provided by one or more of the sensors 61-66, an activation of the hip extension muscles may be done after an activation of the hip flexion, for example, in order to limit the hip flexion movement.

Alternatively or additionally, upon stimulation of the thigh muscles the musculus quadriceps may be activated for the knee extension and the musculus biceps femoris, the musculus semitendinosus and/or the musculus semimembranosus for the knee flexion. Here as well, in addition to the resistance device 30 in the case of an orthotic device 20, a coordinated activation of synergistic and antagonistic muscles may be done together with the resistance device 30. The movement energy from the stimulated muscles is stored temporarily in the orthotic or prosthetic device via the energy accumulator 31 and then furnished to the system once more via the drive unit 32 for assistance, e.g., synergistically to the muscles stimulated.

The activation or stimulation of the muscles occurs via a muscle interface or a nerve interface. Besides a stimulation via an implanted system, as described in the sample embodiments, this may also occur via a surface stimulation system with surface electrodes, as long as the muscles or nerves are accessible from the outside. The mechanical system of the prosthetic and orthotic device 20 controls the performance of the movement via the resistance device 30 in combination with the control device 50 and the sensors 61-66 by adapting the dampening through the resistance device 30 and the blocking or the adjusting of extension end stops and flexion end stops to limit the maximum achievable angle positions.

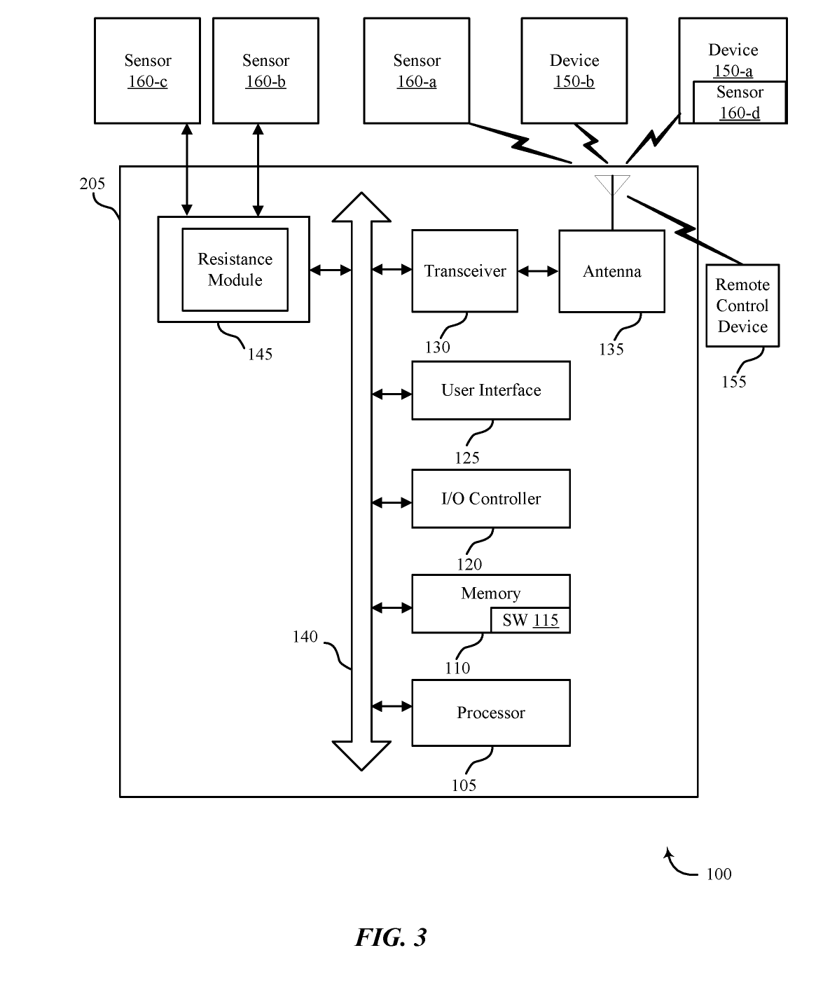

FIG. 3 shows a system 100 for use with the orthotic and prosthetic devices 20 shown in FIGS. 1 and 2. System 100 may include a control panel 205. Control panel 205 may be equivalent at least in part to the control device 50 described above. Control panel 205 may include resistance module 145. The resistance module 145 may provide communications with one or more sensors 160 directly or via other communication components, such as a transceiver 130 and/or antenna 135. The sensors 160 may represent one or more of the sensors 61-66 described above. The resistance module 145 may perform or control various operations associated with, for example, the resistance device 30, energy accumulator 31, drive unit 32, or other components of the orthotic or prosthetic device 20, as described above with reference to FIGS. 1 and 2.

Control panel 205 may also include a processor module 105, and memory 110 (including software/firmware code (SW) 115), an input/output controller module 120, a user interface module 125, a transceiver module 130, and one or more antennas 135 each of which may communicate, directly or indirectly, with one another (e.g., via one or more buses 140). The transceiver module 130 may communicate bi-directionally, via the one or more antennas 135, wired links, and/or wireless links, with one or more networks or remote devices. For example, the transceiver module 130 may communicate bi-directionally with one or more of device 150-a, device 150-b, remote control device 155, and/or sensors 160-a, 160-d. The devices 150-a, 150-b may be components of the orthotic or prosthetic device 20, or other devices in communication with the orthotic or prosthetic device 20. The transceiver module 130 may include a modem to modulate the packets and provide the modulated packets to the one or more antennas 135 for transmission, and to demodulate packets received from the one or more antennas 135. In some embodiments (not shown) the transceiver may be communicate bi-directionally with one or more of device 150-a, device 150-b, remote control device 155, and/or sensors 160-a, 160-d through a hardwired connection without necessarily using antenna 135. While a control panel or a control device (e.g., 205) may include a single antenna 135, the control panel or the control device may also have multiple antennas 135 capable of concurrently transmitting or receiving multiple wired and/or wireless transmissions. In some embodiments, one element of control panel 205 (e.g., one or more antennas 135, transceiver module 130, etc.) may provide a connection using wireless techniques, including digital cellular telephone connection, Cellular Digital Packet Data (CDPD) connection, digital satellite data connection, and/or another connection.

The signals associated with system 100 may include wireless communication signals such as radio frequency, electromagnetics, local area network (LAN), wide area network (WAN), virtual private network (VPN), wireless network (using 802.11, for example), 345 MHz, Z-WAVE®, cellular network (using 3G and/or LTE, for example), and/or other signals. The one or more antennas 135 and/or transceiver module 130 may include or be related to, but are not limited to, WWAN (GSM, CDMA, and WCDMA), WLAN (including BLUETOOTH® and Wi-Fi), WMAN (WiMAX), antennas for mobile communications, antennas for Wireless Personal Area Network (WPAN) applications (including RFID and UWB). In some embodiments, each antenna 135 may receive signals or information specific and/or exclusive to itself. In other embodiments, each antenna 135 may receive signals or information not specific or exclusive to itself.

In some embodiments, one or more sensor units 160 (e.g., angle, velocity, acceleration, force, temperature, etc.) may connect to some element of system 100 via a network using one or more wired and/or wireless connections. In some embodiments, the user interface module 125 may include an audio device, such as an external speaker system, an external display device such as a display screen, and/or an input device (e.g., remote control device interfaced with the user interface module 125 directly and/or through I/O controller module 120).

One or more buses 140 may allow data communication between one or more elements of control panel 205 (e.g., processor module 105, memory 110, I/O controller module 120, user interface module 125, etc.).

The memory 110 may include random access memory (RAM), read only memory (ROM), flash RAM, and/or other types. The memory 110 may store computer-readable, computer-executable software/firmware code 115 including instructions that, when executed, cause the processor module 105 to perform various functions described in this disclosure (e.g., initiating an adjustment of a lighting system, etc.). Alternatively, the software/firmware code 115 may not be directly executable by the processor module 105 but may cause a computer (e.g., when compiled and executed) to perform functions described herein. Alternatively, the computer-readable, computer-executable software/firmware code 115 may not be directly executable by the processor module 105 but may be configured to cause a computer (e.g., when compiled and executed) to perform functions described herein. The processor module 105 may include an intelligent hardware device, e.g., a central processing unit (CPU), a microcontroller, an application-specific integrated circuit (ASIC), etc.

In some embodiments, the memory 110 can contain, among other things, the Basic Input-Output system (BIOS) which may control basic hardware and/or software operation such as the interaction with peripheral components or devices. For example, the resistance module 145, and other modules and operational components of the control panel 205 used to implement the present systems and methods may be stored within the system memory 110. Applications resident with system 100 are generally stored on and accessed via a non-transitory computer readable medium, such as a hard disk drive or other storage medium. Additionally, applications can be in the form of electronic signals modulated in accordance with the application and data communication technology when accessed via a network interface (e.g., transceiver module 130, one or more antennas 135, etc.).

Many other devices and/or subsystems may be connected to one or may be included as one or more elements of system 100. In some embodiments, all of the elements shown in FIG. 3 need not be present to practice the present systems and methods. The devices and subsystems can be interconnected in different ways from that shown in FIG. 3. In some embodiments, an aspect of some operation of a system, such as that shown in FIG. 3, may be readily known in the art and are not discussed in detail in this application. Code to implement the present disclosure can be stored in a non-transitory computer-readable medium such as one or more of system memory 110 or other memory. The operating system provided on I/O controller module 120 may be iOS®, ANDROID®, MS-DOS®, MS-WINDOWS®, OS/2®, UNIX®, LINUX®, or another known operating system.

The transceiver module 130 may include a modem configured to modulate the packets and provide the modulated packets to the antennas 135 for transmission and/or to demodulate packets received from the antennas 135. While the control panel or control device (e.g., 205) may include a single antenna 135, the control panel or control device (e.g., 205) may have multiple antennas 135 capable of concurrently transmitting and/or receiving multiple wireless transmissions.

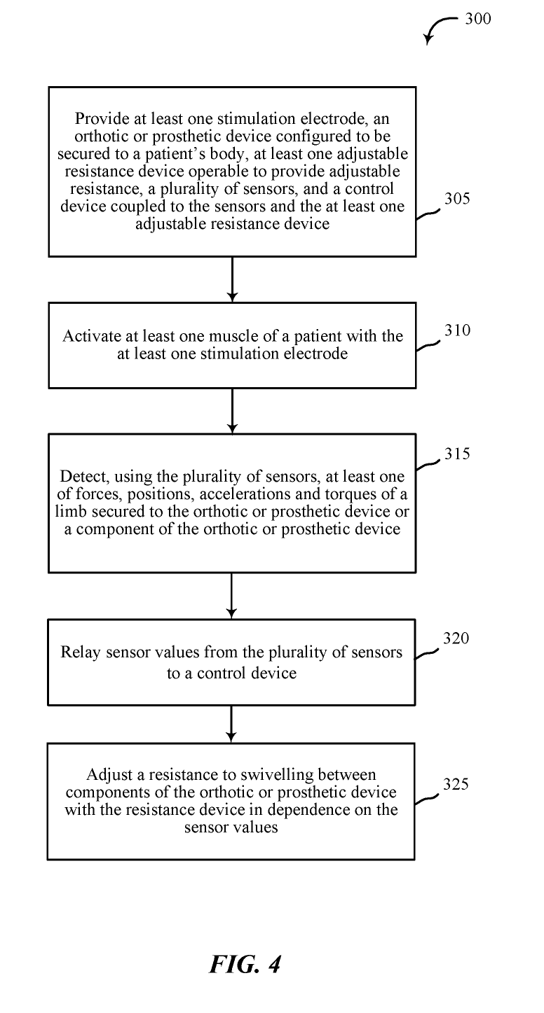

FIG. 4 is a flow chart illustrating an example of a method 300 for improving orthotic or prosthetic devices, in accordance with various aspects of the present disclosure. One or more aspects of the method 300 may be implemented in conjunction with orthotic or prosthetic device 20 of FIGS. 1 and 2, resistance module 145 depicted in FIG. 3, and/or control panel 205 shown in FIG. 3. In some examples, a computing device may execute one or more sets of codes to control the functional elements of the computing device or aspects of the orthotic or prosthetic device 20, resistance module 145, and/or control panel 205 to perform one or more of the functions described below. Additionally or alternatively, the computing device may perform one or more of the functions described below using special-purpose hardware.

At block 305, method 300 may include providing at least one stimulation electrode, an orthotic or prosthetic device configured to be secured to a patient's body, at least one adjustable resistance device operable to provide adjustable resistance, a plurality of sensors, and a control device coupled to the sensors and the at least one adjustable resistance device. At block 310, the method 300 may include activating at least one muscle of a patient with the at least one stimulation electrode. Block 315 may include detecting, using the plurality of sensors, at least one of forces, positions, accelerations and torques of a limb secured to the orthotic or prosthetic device or a component of the orthotic or prosthetic device. Block 320 may include relaying sensor values from the plurality of sensors to a control device. Method 300 may also include, at block 325, adjusting a resistance to swivelling between components of the orthotic or prosthetic device with the resistance device in dependence on the sensor values.

In other embodiments, methods according to the present disclosure may include further steps in addition to those shown in FIG. 4. In some embodiments, the methods may include variations of the steps shown in FIG. 4, or few steps than those shown in FIG. 4.

The detailed description set forth above in connection with the appended drawings describes examples and does not represent the only instances that may be implemented or that are within the scope of the claims. The terms “example” and “exemplary,” when used in this description, mean “serving as an example, instance, or illustration,” and not “preferred” or “advantageous over other examples.” The detailed description includes specific details for the purpose of providing an understanding of the described techniques. These techniques, however, may be practiced without these specific details. In some instances, known structures and apparatuses are shown in block diagram form in order to avoid obscuring the concepts of the described examples.

Information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and components described in connection with this disclosure may be implemented or performed with a general-purpose processor, a digital signal processor (DSP), an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, and/or state machine. A processor may also be implemented as a combination of computing devices, for example, a combination of a DSP and a microprocessor, multiple microprocessors, one or more microprocessors in conjunction with a DSP core, and/or any combination thereof.

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope and spirit of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations.

As used herein, including in the claims, the term “and/or,” when used in a list of two or more items, means that any one of the listed items can be employed by itself, or any combination of two or more of the listed items can be employed. For example, if a composition is described as containing components A, B, and/or C, the composition can contain A alone; B alone; C alone; A and B in combination; A and C in combination; B and C in combination; or A, B, and C in combination. Also, as used herein, including in the claims, “or” as used in a list of items (for example, a list of items prefaced by a phrase such as “at least one of” or “one or more of”) indicates a disjunctive list such that, for example, a list of “at least one of A, B, or C” means A or B or C or AB or AC or BC or ABC, or A and B and C.

In addition, any disclosure of components contained within other components or separate from other components should be considered exemplary because multiple other architectures may potentially be implemented to achieve the same functionality, including incorporating all, most, and/or some elements as part of one or more unitary structures and/or separate structures.

Computer-readable media includes both computer storage media and communication media including any medium that facilitates transfer of a computer program from one place to another. A storage medium may be any available medium that can be accessed by a general purpose or special purpose computer. By way of example, and not limitation, computer-readable media can comprise RAM, ROM, EEPROM, flash memory, CD-ROM, DVD, or other optical disk storage, magnetic disk storage or other magnetic storage devices, or any other medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, or any combination thereof, then the coaxial cable, fiber optic cable, twisted pair, DSL, or wireless technologies such as infrared, radio, and/or microwave are included in the definition of medium. Disk and disc, as used herein, include any combination of compact disc (CD), laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The previous description of the disclosure is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not to be limited to the examples and designs described herein but is to be accorded the broadest scope consistent with the principles and novel features disclosed.

The process parameters, actions, and steps described and/or illustrated in this disclosure are given by way of example only and can be varied as desired. For example, while the steps illustrated and/or described may be shown or discussed in a particular order, these steps do not necessarily need to be performed in the order illustrated or discussed. The various exemplary methods described and/or illustrated here may also omit one or more of the steps described or illustrated here or include additional steps in addition to those disclosed.

Furthermore, while various embodiments have been described and/or illustrated here in the context of fully functional computing systems, one or more of these exemplary embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may permit and/or instruct a computing system to perform one or more of the exemplary embodiments disclosed here.

This description, for purposes of explanation, has been described with reference to specific embodiments. The illustrative discussions above, however, are not intended to be exhaustive or limit the present systems and methods to the precise forms discussed. Many modifications and variations are possible in view of the above teachings. The embodiments were chosen and described in order to explain the principles of the present systems and methods and their practical applications, to enable others skilled in the art to utilize the present systems, apparatus, and methods and various embodiments with various modifications as may be suited to the particular use contemplated.

We claim:

1. A orthopedic system, comprising:
at least one stimulation electrode to activate at least one muscle;
an orthotic or prosthetic device configured to be secured to a patient's body, the orthotic or prosthetic device comprising:
a proximal component;
a distal component;
at least one joint connecting the proximal and distal components to each other with a swivel connection;
at least one adjustable resistance device arranged between the distal and proximal components and operable to provide adjustable resistance against relative swivelling between the proximal and distal components;
a plurality of sensors to detect at least one of forces, positions, accelerations and torques;
a control device coupled to the sensors and the at least one adjustable resistance device, the control device processing sensor values received from the sensors and adjusting the at least one adjustable resistance device based on the sensor values.

2. The orthopedic system according to claim 1, wherein the at least one stimulation electrode is designed as a surface electrode or as an implant.

3. The orthopedic system according to claim 1, wherein the at least one stimulation electrode is coupled to the control device.

4. The orthopedic system according to claim 1, further comprising an energy accumulator operable to store kinetic energy of the orthotic or prosthetic device and return the stored kinetic energy to the orthotic or prosthetic device in a controlled manner.

5. The orthopedic system according to claim 4, wherein the energy accumulator comprises a spring accumulator, pressure accumulator, or accumulator of electric energy.

6. The orthopedic system according to claim 1, further comprising a drive unit associated with the orthotic or prosthetic device.

7. The orthopedic system according to claim 1, wherein the orthotic or prosthetic device is a lower limb orthotic or prosthetic device.

8. The orthopedic system according to claim 1, wherein the at least one adjustable resistance device comprises a mechanical brake, a fluid damper, a magnetorheological damper, or a generator.

9. A method for control of an orthopedic system, comprising:
providing at least one stimulation electrode, an orthotic or prosthetic device configured to be secured to a patient's body, at least one adjustable resistance device operable to provide adjustable resistance, a plurality of sensors, and a control device coupled to the sensors and the at least one adjustable resistance device;
activating at least one muscle of a patient with the at least one stimulation electrode;
detecting, using the plurality of sensors, at least one of forces, positions, accelerations and torques of a limb secured to the orthotic or prosthetic device or a component of the orthotic or prosthetic device;
relaying sensor values from the plurality of sensors to a control device;
adjusting a resistance to swivelling between components of the orthotic or prosthetic device with the at least one adjustable resistance device in dependence on the sensor values.

10. The method according to claim 9, wherein the orthotic or prosthetic device comprises a proximal component, a distal component, and at least one joint connecting the proximal and distal components to each other with a swivel connection, and the at least one adjustable resistance device adjusts a resistance to swivelling between the proximal and distal components.

11. The method according to claim 9, wherein activating at least one muscle includes activating muscles which act antagonistically to each other by at least two stimulation electrodes delayed in time.

12. The method according to claim 9, further comprising providing a drive unit, the drive unit assisting a swivelling movement of a proximal component relative to a distal component.

13. The method according to claim 9, further comprising storing movement energy from the activated muscles in an energy accumulator and returning the stored movement energy to drive the orthotic or prosthetic device.

14. The method according to claim 9, wherein activating the at least one muscle includes activation in dependence on a force exerted on a limb or a component of the orthotic or prosthetic device or a torque exerted on a limb or a component of the orthotic or prosthetic device.

15. The method according to claim 9, wherein activating the at least one muscle includes activation in dependence on at least one of an acceleration performed by a limb or a component of the orthotic or prosthetic device, or on a position of a limb or a component of the orthotic or prosthetic device in space or relative to each other.

16. The method according to claim 9, wherein activating the at least one muscle includes activation of at least one of hip muscles and thigh muscles.
